(19) United States
(12) Patent Application Publication
(10) Pub. No.: US 2003/0161762 A1
(43) Pub. Date: Aug. 28, 2003

(54) Instrument for determining concentration of multiple analytes in a fluid sample

(76) Inventors: Michael Caron, Indianapolis, IN (US); Matthew Wallace, Ft. Wayne, IN (US)

Correspondence Address: Michael C. Bartol, 111 Monument Circle, Suite 4600, P.O. Box 44924, Indianapolis, IN, 46244, US

(21) Appl. No.: 10/361,779

(22) Filed: Feb. 10, 2003

Related U.S. Application Data
(60) Provisional application No. 60/355,165, filed on Feb. 8, 2002.

(51) Int. Cl. C12M 1/34; H04K 1/00; G01N 33/00

(52) U.S. Cl. 422/68.1; 435/287.2; 380/2

(57) ABSTRACT

An instrument for reading test strips to determine concentrations of analytes from body fluid samples that are deposited onto the test strips. The instrument includes a novel modular optical block that can be replaced without requiring replacement of the entire instrument. Another feature of the invention is a novel encryption code that allows a technician during service calls to quickly identify all relevant manufacturing information for the test strips being used by the person placing the service call. Another feature of the instrument of the present invention is that it can be used in combination with a novel test strip capable of testing multiple analytes from a single sample.

### RELATED APPLICATIONS

This application claims priority from U.S. provisional application serial No. 60/355,165, filed Feb. 8, 2002.

### FIELD OF THE INVENTION

The present invention relates generally to testing of body fluids for concentration of analytes and more particularly to an instrument that receives test strips and measures the concentration of analytes from fluids deposited on the test strips.

### BACKGROUND

The level of certain analytes in blood and other body fluids can predict disease or risk thereof. For example, cholesterol in blood is a significant indicator of risk of coronary heart disease. “Total cholesterol” includes low density lipoproteins (LDL), very low density lipoproteins (VLDL) and high density lipoproteins (HDL). It is well established from epidemiological and clinical studies that there is a positive correlation between levels of LDL and VLDL cholesterol (“bad” cholesterol) and coronary heart disease and a negative correlation between levels of HDL cholesterol (“good” cholesterol) and coronary heart disease. The level of total cholesterol in blood, which is a measure of the sum total of HDL, LDL, VLDL and chylomicrons, is not generally regarded as an adequate indicator of the risk of coronary heart disease because the overall level of total cholesterol does not reveal the relative proportions of HDL, LDL and VLDL. To better assess the risk of heart disease, it is desirable to determine the amount of HDL, LDL and triglycerides in addition to total cholesterol.

U.S. Pat. No. 5,597,532 discloses an apparatus for optoelectronic evaluation of test strips for use in the detection of certain analytes in blood or other body fluids. The test strip used with such instrument comprises an elongated plastic part including a hinged portion to allow a first portion to be folded over a second portion. A series of test strip layers are disposed between the folded over portions of the test strip. The method involves providing a separately colored strip and corresponding memory module for each test. For example, total cholesterol strips and modules may be colored red, whereas glucose strips and modules may be colored yellow, and so forth. However, a separate sample must be used and a separate test conducted for each analyte for which concentration is to be determined.

Devices known to applicants for measuring multiple analytes in a single sample are complex. For example, one known device to measure the concentration of HDL cholesterol and other analytes from a whole blood sample is disclosed in U.S. Pat. No. 5,213,965 (Jones) and other related and commonly assigned patents. The device includes a well in which the whole blood sample is deposited and then drawn through a capillary to a sieving pad made of fibrous material. The sieving pad achieves initial separation of blood cells from plasma on the basis of the blood cell's slower migration rate therethrough. The sieving pad is covered with a microporous membrane which further filters blood cells. Covering the microporous membrane is a reagent reservoir membrane containing precipitating agents for LDL and VLDL on one side thereof. On the other side of the reagent reservoir, there are no precipitating agents.

On top of and extending laterally beyond the reagent reservoir is an elongate matrix which distributes the sample laterally after it leaves the reservoir. Finally, one or more test pads are positioned above and biased apart from the elongate matrix. Plasma exits the filtering membrane and enters the reagent reservoir where LDL and VLDL cholesterol are precipitated on one side thereof and then flow from the reservoir and migrate laterally through one side of the elongate matrix. Similarly, plasma that enters the other side of the reagent reservoir encounters no precipitating agents, and this plasma exits the side of the elongate matrix opposite the side the plasma containing precipitated LDL and VLDL cholesterol exits. At a desired time, the test pads can be depressed so they are in fluid communication with the elongate matrix. The test pads that contact one side of the elongate matrix measure concentration of HDL, whereas the test pads that contact the opposite side of the elongate matrix measure total cholesterol.

Undesirably, the test pads must be kept spaced apart from the elongate matrix until the entire operation is properly timed, whereupon the test plate having the test pads thereon can be depressed against the elongate matrix. Of course, manually depressing the test pad creates a process step that must be accomplished by hand or by mechanical means within the instrument.

An undesirable drawback of presently known instruments that read test strips is that the area or port in which the test strip is received becomes contaminated by residual blood or fluid sample which escapes the confines of the test strip. The port must be cleaned frequently and often becomes contaminated to the extent that test results can be compromised. Similarly, the port typically contains a glass window which is susceptible to breakage. Thus, an otherwise functioning test strip instrument may need to be replaced merely because the port has been irreversibly contaminated or broken.

Another undesirable drawback of presently known instruments that read test strips is that technicians responding to service calls often cannot readily identify all of the pertinent manufacturing information regarding the test strips that the caller is using.

It would be desirable to overcome the drawbacks of the prior art noted above and provide a test strip diagnostic instrument that is generally easier to use and provides enhanced functionality.

### SUMMARY OF THE INVENTION

The present invention provides an instrument for determining concentrations of analytes from body fluid samples that are deposited onto test strips. The instrument includes a novel modular optical block that can be replaced without requiring replacement of the entire instrument. Another feature of the invention is a novel encryption code that allows a technician during service calls to quickly identify all relevant manufacturing information for the test strips being used by the person placing the service call. Another feature of the instrument of the present invention is that it can be used in combination with a novel test strip capable of testing multiple analytes from a single sample. The instrument reads the color density of multiple analytes and displays the same on an easy-to-read display.

### BRIEF DESCRIPTION OF DRAWINGS

The above-mentioned and other advantages of the present invention, and the manner of obtaining them, will become more apparent and the invention itself will be better understood by reference to the following description of the embodiments of the invention taken in conjunction with the accompanying drawings, wherein:

FIG. 1 is an exploded perspective view showing the instrument, optical block and test strip in accordance with the present invention;

FIG. 2 is an exploded perspective view of a test strip holder in accordance with the present invention illustrating a test matrix and its relationship with the top and bottom portions of the test strip holder;

FIG. 3 is a side sectional view of an exemplary test matrix in accordance with one embodiment of the present invention;

FIG. 4 is a side sectional view of an exemplary test matrix in accordance with another embodiment of the present invention;

FIG. 5 is a block diagram schematic view illustrating the parts and operation of the test instrument in accordance with the present invention; and

FIG. 6 is a diagrammatic illustration of a display for the instrument of the present invention that illustrates a novel encryption scheme.

Corresponding reference characters indicate corresponding parts throughout the several views.

### DETAILED DESCRIPTION

The embodiments of the present invention described below are not intended to be exhaustive or to limit the invention to the precise forms disclosed in the following detailed description. Rather, the embodiments are chosen and described so that others skilled in the art may appreciate and understand the principles and practices of the present invention.

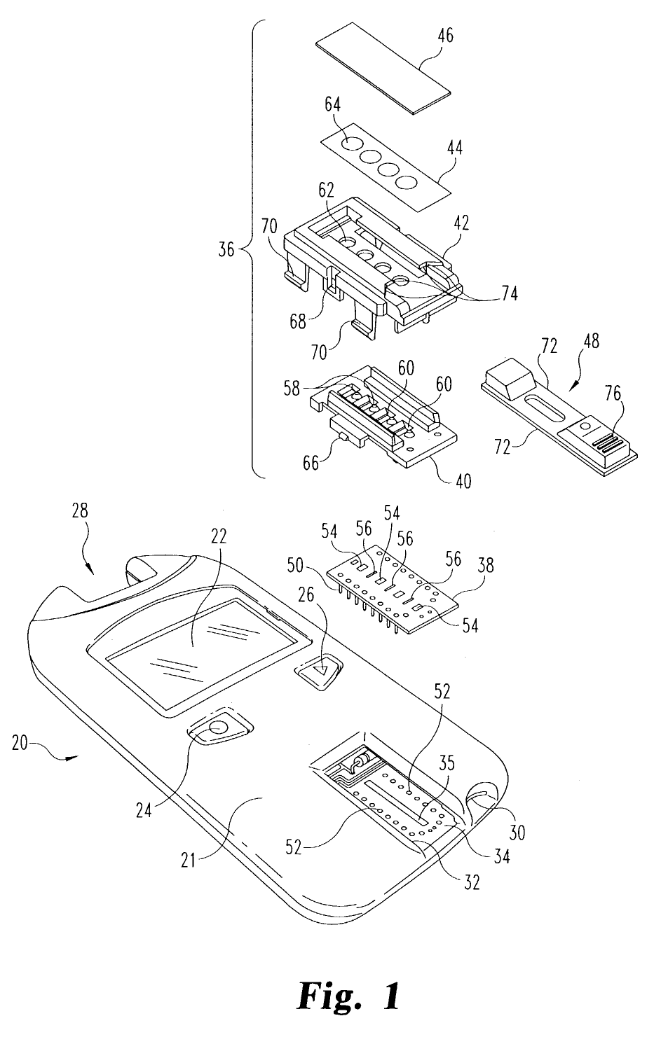

Referring now to FIG. 1, instrument 20 includes instrument body 21, display 22, keys 24 and 26, port 28, which receives a Memo chip (not shown), port 30 for an amperometric test strip (not shown), and opening 32. In the inside of instrument 20 is the main circuit board 34 and compression spacer 35, part of which can be seen in opening 32. Optical block 36 includes optical hybrid chip 38, light shield 40, strip holder 42, die cut adhesive 44, and glass 46. Finally, a test strip 48 is inserted into holder 42.

Still referring to FIG. 1, chip 38 has pins 50 that are received into holes 52 in circuit board 34 by soldering or other fastening means known in the art. Optical Hybrid chip 38 includes photodiodes 54 and LED arrays 56. Photodiodes 54 align with openings or pores 58 in light shield 40 while LED arrays 56 align with rectangular openings 60 in light shield 40. In turn, pores 58 align with the direct center of respective holes 62 and 64 in strip holder 42 and adhesive 44, respectively. Rectangular openings 60 align with the sides of holes 62 and 64 so that the incident beam is directed to the relevant portions of test strip 48 at an angle, as explained below.

Conveniently, optical hybrid chip 38 is first positioned and inserted into main circuit board 34 on top of compression spacer 3. In turn, light shield 40 snaps into strip holder 42 by means of tabs 66 that are received in corresponding openings 68 as shown. Glass 46 is secured to strip holder 42 by adhesive 44 or other suitable means known in the art. The entire optical block assembly 36, once fastened together, snaps into the main circuit board 34 by means of legs 70 that bias outwardly and engage the side of an oblong opening in the main circuit board 34. Compression spacer 35 serves the purpose of compressing the optical hybrid chip 38 into light shield 40 to maintain minimal deviation of mechanical stack-up in the optical block assembly. After the optical block subassembly is firmly affixed into main circuit board 34, optical hybrid chip 38 is soldered into place on the opposite side (not shown). The optical block of the present invention is advantageous because the optical hybrid chip 38 can be assembled separately and by a different facility that circuit board 34, thereby saving time and manufacturing costs. Another advantage of the optical block is that it can be replaced as a single unit without requiring replacement of the entire instrument. Still another advantage of the optical block is that its modular design allows interchangeable optical blocks with different capabilities.

When installed in instrument 20, holder 42 of optical block 36 receives test strip 48. More specifically, sides 72 of test strip 48 are fittingly received into grooves 74 of holder 42. A handle portion 76 aids the user in inserting the strips 48 into the instrument.

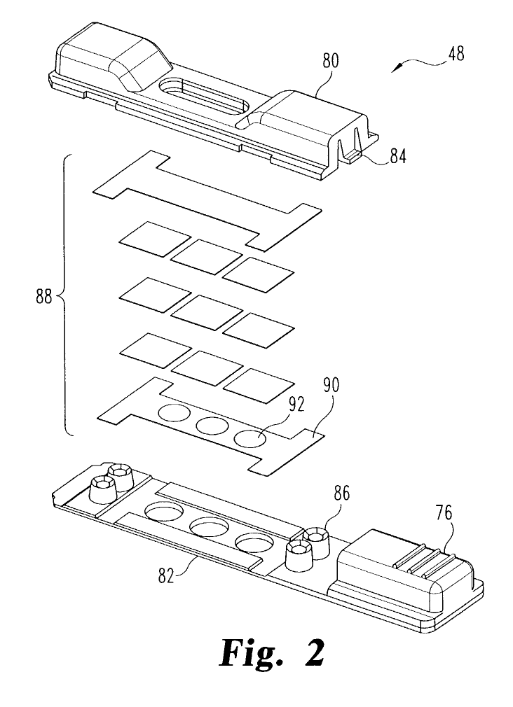

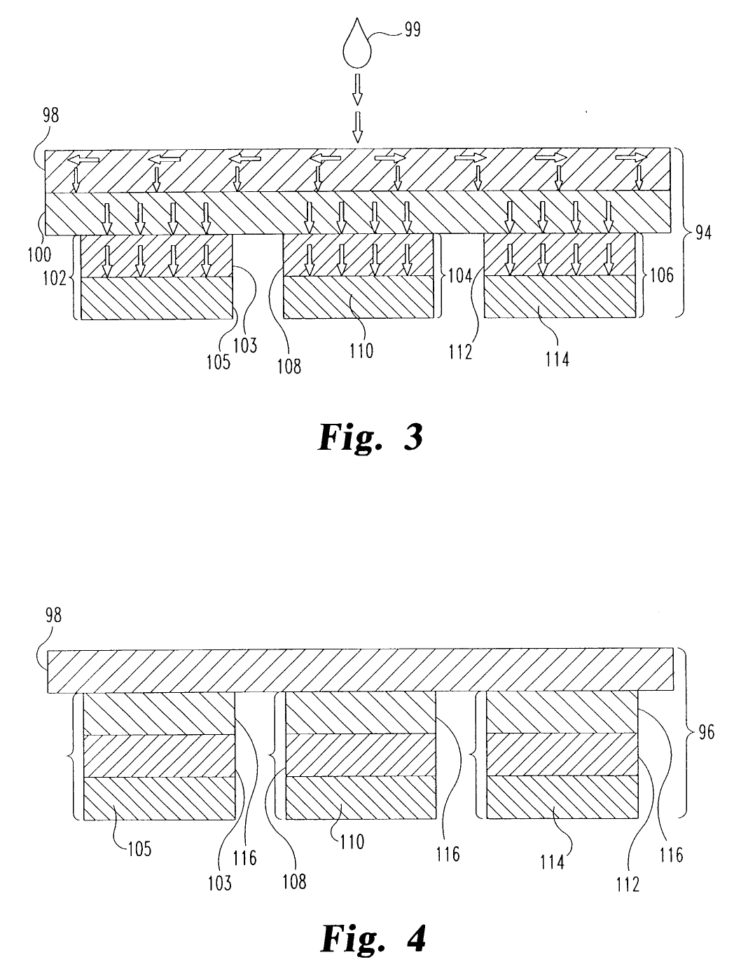

Turning now to FIG. 2, test strip 48 is preferably formed by injection molding. Test strip 48 includes handle 76 and top portion 80, which is preferably hingedly attached to bottom portion 82. Top portion 80 includes a leg member 84 that is inserted into a corresponding opening (not shown) in portion 82 and thereby secures top portion 80 to bottom portion 82. The other side of top portion 80, as mentioned, is preferably hingedly attached to bottom portion 82. Bosses 86 receive complementary pegs (not shown) that extend downwardly from top portion 80 and produce a snap-fit engagement of top portion 80 to bottom portion 82. Test matrix 88 is described with reference to FIGS. 3 and 4, except to note with reference to FIG. 2 that adhesive layer 90 having openings 92 is used to hold the matrix together during assembly.

Turning now to FIGS. 3 and 4, two different test matrices 94 and 96 are illustrated. Both matrices 94 and 96 include top disbursement layer 98. Layer 98 is an open cell layer capable of rapidly and effectively spreading the fluid sample. One suitable material for layer 98 is available under the name “Accuflow Plus-P,” Schleicher & Schuell, Inc. Another suitable material for layer 98 is available under the name “Accuwik,” Pall Biochemicals. Both of these layers are made of polyester and provide excellent movement of blood therethrough as shown by the arrows in FIG. 3.

Layer 100 is a blood separation layer that is adjacent to and in fluid communication with layer 98. Blood separation layer 100 separates a portion of blood cells from plasma and passes blood filtrate therethrough. A suitable commercial membrane for layer 40 is Ahlstrom Grade 144, thickness 0.378 mm, available from Ahlstrom Filtration, Inc., Mt. Holly Springs, Pa.

Below and in fluid communication with layer 100 are three “stacks.” For example, stack 102 can be an HDL Measurement Stack that includes layers 104 and 106. Stack 104 can be a total cholesterol stack including layers 108 and 110. Finally, stack 106 can be a triglyceride stack including layers 112 and 114. Importantly, it should be understood that the “stacks” can be other than the examples just noted. For example, the stacks can measure, e.g., ketones, glucose, creatinine, and other body fluids. Further, more or less than the two layers shown for the stacks 102, 104 and 106 can be used, depending upon the particular analyte to be measured. With reference to FIG. 4, matrix 96 includes disbursement layer 98, as does matrix 94. However, blood separation layer 100 has been replaced with individual blood separation layers 116.

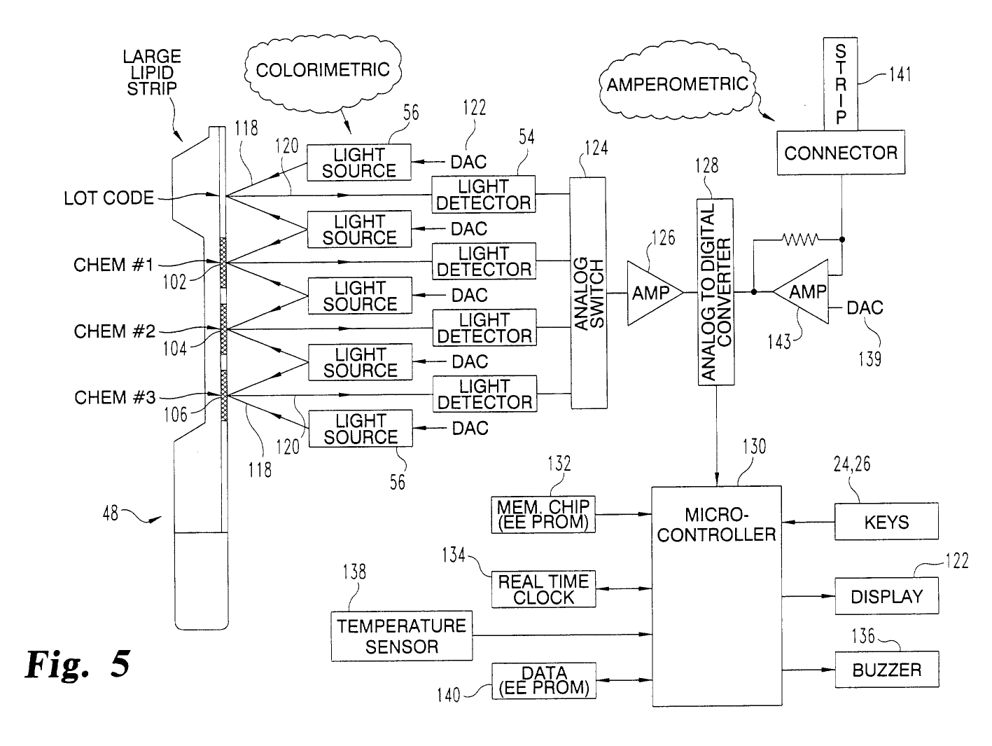

Turning now to FIG. 5, the operation of the instrument can be better understood. Once test strip 48 is inserted into test strip holder 42, photodiodes 54 and LED arrays (or light sources) 56 align as shown so that the incident light 118 is directed towards the bottom reaction layer of the various stacks 102, 104 or 106 at an angle as depicted in FIG. 5. The reflected light 120 reflects directly back through pores 58 (see FIG. 1) and is measured by light detectors or photodiodes 54. Digital to Analog Voltage Converters 122 drive the light sources based upon a predetermined calibration value. To more fully explain, the optical loop of the instrument is calibrated such that white corresponds to 100% reflectance and black corresponds to 0% reflectance. The light sources or LED arrays can be configured to emit green, red, blue or other colored lights depending upon the color of the reaction membrane to be measured. For example, cholesterol reaction membranes produce a blue color, such that a red LED has been found to work best to measure reflectance.

The light detectors produce a very small current that is amplified and converted into a voltage which is then multiplexed by analog switch 124 into one of two amplifier stages 126 depending upon which LED is used. The output signal from amplifier stage 126 is then input into analog to digital converter 128 and converted into a digital signal for acquisition by microcontroller 130.

The instrument is essentially a closed loop control system, in that micro-controller 130 drives DAC's 122 and then reads the signal that is returned from converter 128. Memo chip 132 is an interchangeable part that fits into port 28 (FIG. 1). The Memo chip contains information concerning the particular container of test strips that is to be used with the instrument, such as lot code, expiration date, which of the LEDs 56 to light (e.g., blue or red), the chemistry curve which correlates the reflectance to analyte concentration, etc. As shown in the upper left hand corner of FIG. 5, the lot code can be correlated to the color of the test strip, or shade of color. For example, if a green test strip is inserted into the instrument, and the Memo chip requires a yellow test strip, an error code would be displayed on display 22 (FIG. 1). Such a system is described, for example, in U.S. Pat. No. 5,597,532.

Real time clock 134 maintains the time and date information for controller 130. Micro-controller 130 sends a signal to buzzer 136 which in turn produces an audible sound when there is an error or warning signal. Keys 24 and 26 (also see FIG. 1) allow the user to operate the instrument 20. Temperature sensor 138 inputs ambient temperature information to micro-controller 130, whereby the micro-controller will not allow the instrument to perform chemistry tests if the sensed temperature is outside of a pre-determined range. Finally, data EEPROM 140 is a general storage device that stores information such as chemistry results and other parameters that are generated, for example, during calibration.

With reference to the upper right-hand corner of FIG. 5, the amperometric testing functions when the DAC 139 applys a voltage across the strip 141 (resistive sensor) which in turn generates a current when dosed with whole blood. The current is proportional to the concentration of glucose, if that happens to be the analyte being tested. The current is amplified by amp 143 and converted into a voltage and input into the Analog-to-Digital Converter 128, which in turn converts the signal into a binary value which can be read by micro-controller 130. Micro-controller 130 processes the information and sends the result to display 22, which then displays the concentration of analyte.

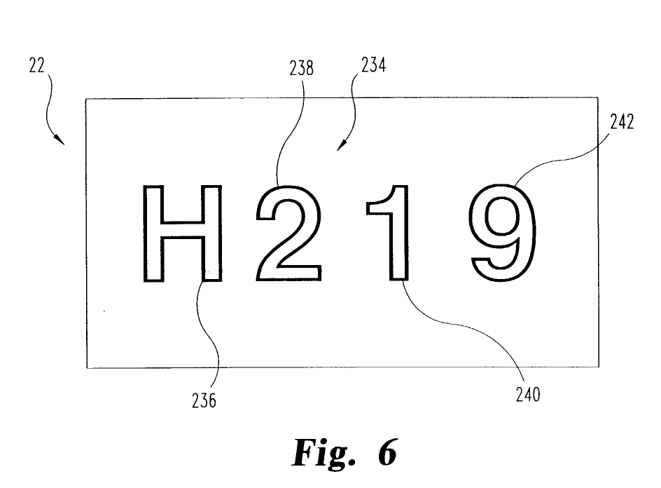

Another aspect of the present invention relates to a code which flashes on display 22 after Memo chip 132 (FIG. 5) is installed into port 28 (FIG. 1) and the “Run Test” mode is selected. As shown in FIG. 6, display 22 has a four-digit code 134 consisting of first digit 236, second digit 238, third digit 240 and fourth digit 242. In the exemplary embodiment, digit 236 is an alphanumeric character that corresponds to the chemistry, in this case “H” for HDL. The second digit 238 is an alphanumeric character that corresponds to the year in which the test strip was made, in this case “2” for 2002. The third digit 240 is an alphanumeric character that corresponds to the month, in this case “1” for January. Finally, the fourth digit 242 is an alphanumeric character that corresponds to the sequential lot or batch from that particular month in which the test strip was made. Such a code presents a convenient way for a technician at the company which produces the instruments and strips to quickly identify all relevant manufacturing information regarding a particular test strip with only four alphanumeric characters. Such an encryption scheme saves time and helps the technician more quickly identify the source of a malfunction of the system during service calls.

While a preferred embodiment incorporating the principles of the present invention has been disclosed hereinabove, the present invention is not limited to the disclosed embodiments. Instead, this application is intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. An instrument for measuring the concentration of analytes, comprising:
an instrument body defining an opening; and
a modular optical block detachably received in said opening, said optical block comprising a test strip holder adapted to receive a test strip, whereby said modular optical block can be replaced without requiring replacement of said instrument.

2. The instrument of claim 1, wherein said optical block further comprises a light shield which directs incident beams toward the test strips that are received in said test strip holder and directs reflected beams from the test strips to a light detector.

3. The instrument of claim 2, further comprising a chip attached to the underside of said light shield, said chip having said light detectors mounted thereon and having light sources mounted thereon, said light sources producing said incident beams.

4. An encryption scheme for a diagnostic instrument that reads test strips, said encryption scheme comprising:
a display having four alphanumeric characters, a first one of said alphanumeric characters corresponding to the type of analyte the test strip measures, the second alphanumeric character corresponding to the year the test strip was made, the third alphanumeric character corresponding to the month the test strip was made, and the fourth alphanumeric character corresponding to a lot or batch number of the test strip.
